(19) United States
(12) Patent Application Publication
(10) Pub. No.: US 2025/0065124 A1
(43) Pub. Date: Feb. 27, 2025

(54) METHODS FOR EVOKED RESPONSES

(71) Applicant: Boston Scientific Neuromodulaton Corporation, Valencia, CA (US)

(72) Inventors: G. Karl Steinke, Valencia, CA (US); Tomasz Mark Fraczek, Yucaipa, CA (US)

(21) Appl. No.: 18/810,207

(22) Filed: Aug. 20, 2024

Related U.S. Application Data
(60) Provisional application No. 63/534,623, filed on Aug. 25, 2023.

(51) Int. Cl. A61N 1/36; A61N 1/05; A61N 1/372

(52) U.S. Cl. CPC A61N 1/36128; A61N 1/0534; A61N 1/37247

(57) ABSTRACT

A system may include a stimulus circuit configured to provide electrostimulation to a neural target of a patient via electrodes on a lead, a sensing circuit configured to sense evoked response (ER) signals produced by the electrostimulation, a user interface, and a controller operably connected to the stimulus circuit, the sensing circuit, and the user interface. The controller is configured to initiate delivery of the electrostimulation to the electrodes, identify ER signal features of the sensed ER signals, compute a longitudinal distribution of the ER signal features for a longitudinal direction of the lead, compute a rotational distribution that is a is a periodic distribution of the ER signal features for an angular direction of the lead, and display peak regions of the longitudinal distribution and the rotational distribution as a hotspot view of the sensed ER signals on the user interface.

### CLAIM OF PRIORITY

This application claims the benefit of U.S. Provisional Application No. 63/534,623, filed on Aug. 25, 2023, which is hereby incorporated by reference in its entirety.

### TECHNICAL FIELD

This document relates generally to medical systems, and more particularly, but not by way of limitation, to systems, devices, and methods for using sensed evoked responses to guide neurostimulation.

### BACKGROUND

Medical devices may include therapy-delivery devices configured to deliver a therapy to a patient or subject and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

It has been proposed to use evoked potentials as feedback for neurostimulation lead placement and/or neurostimulation programming. For example, Evoked Resonant Neural Activity (ERNA) has been proposed as a guiding or feedback signal for STN DBS therapy for Parkinson's disease. (See Thevathasan W, Sinclair N C, Bulluss K J and McDermott H J (2020) Tailoring Subthalamic Nucleus Deep Brain Stimulation for Parkinson's Disease Using Evoked Resonant Neural Activity. Front. Hum. Neurosci. 14:71. doi: 10.3389/fnhum.2020.00071.) ERNA may also be referred to by other names such as DBS Local Evoked Potentials (DLEP). Evoked potentials, including ERNA or DLEPs, may be present in other indications and anatomical structures or locations. It is desired to improve the lead placement and/or programming using ERNA or other evoked responses.

### SUMMARY

Electrical stimulation, or electrostimulation, can be provided to a patient using a lead that includes multiple electrodes. The electrostimulation can be steered toward a target by allocating the stimulation energy to a specific combination of the electrodes. The present inventors have recognized that it can be challenging to steer the electrostimulation to the desired evoked response target or to achieve the desired evoked response. Embodiments of the present subject matter provide systems, device, and methods that improve steering of electrostimulation energy using feedback from ER signals.

Example 1 includes subject matter (such as a computer-implemented method of operating a neurostimulation system when connected to electrodes of a lead) comprising delivering neurostimulation to a subject using a stimulus circuit, producing sensed evoked response (ER) signals using a sensing circuit, extracting ER signal features from the ER signals using a controller, computing a longitudinal distribution of the ER signal features for a longitudinal direction of the lead using the controller, computing a periodic rotational distribution of the ER signal features for an angular direction of the lead using the controller, determining a peak region of the computed longitudinal distribution and a peak region of the computed periodic rotational distribution of the sensed ER signals, and presenting the peak regions as a hotspot view of the sensed ER signals on a user interface.

In Example 2, the subject matter of Example 1 optionally includes detecting when the peak region of the computed periodic rotational distribution is within a specified range of a circular boundary of the periodic rotational distribution, computing an angular rotation of the periodic rotational distribution to move the peak region farther away from the circular boundary, and determine the hotspot view using the computed angular rotation in response to the detecting.

In Example 3, the subject matter of one or both of Examples 1 and 2 optionally includes computing a wrapped fit function of the ER signal features.

In Example 4, the subject matter of Example 3 optionally includes computing a wrapped gaussian distribution and computing a non-periodic gaussian distribution of the ER signal features.

In Example 5, the subject matter of one or any combination of Examples 1-4 optionally includes displaying a hotspot indicator superimposed on representations of electrodes of the lead on the user interface.

In Example 6, the subject matter of one or any combination of Examples 1-5 optionally includes displaying: a longitudinal location of the peak regions on the length of the lead, an angular location of the peak regions about the lead, and a width of the peak regions at the length location and the angular location.

In Example 7, the subject matter of one or any combination of Examples 1-6 optionally includes displaying representations of electrodes of the lead on the user interface, and displaying the longitudinal distribution and the periodic rotational distribution of the one or more extracted signal features on the user interface with the representations of electrodes of the lead.

In Example 8, the subject matter of one or any combination of Examples 1-7 optionally includes a stimulus circuit configured to provide electrostimulation to electrodes of a deep brain stimulator (DBS) lead, and the evoked response signals produced by the electrostimulation include Evoked Resonant Neural Activity (ERNA) signals.

Example 9 includes subject matter (such as a system) or can optionally be combined with one or any combination of Examples 1-8 to include such subject matter, comprising a stimulus circuit configured to provide electrostimulation to a neural target of a patient via electrodes on a lead, a sensing circuit configured to sense, at a plurality of sensing locations, evoked response (ER) signals produced by the electrostimulation, a user interface, and a controller operably connected to the stimulus circuit, the sensing circuit and the user interface. The controller is configured to initiate delivery of the electrostimulation to the electrodes, identify ER signal features of the sensed ER signals, compute a longitudinal distribution of the ER signal features for a longitudinal direction of the lead, compute a periodic rotational distribution of the ER signal features for an angular direction of the lead, wherein the rotational distribution is a periodic rotational distribution, and display peak regions of the longitudinal distribution and the periodic rotational distribution as a hotspot view of the sensed ER signals on the user interface.

In example 10, the subject matter of Example 9 optionally includes a controller configured to determine when the peak region of the computed periodic rotational distribution is within a specified range of a circular boundary of the periodic rotational distribution, and compute an angular rotation of the periodic rotational distribution to adjust the peak region for the periodic rotational distribution.

In Example 11, the subject matter of one or both of Examples 9 and 10 optionally includes a controller configured to compute a non-periodic gaussian distribution for the longitudinal distribution and compute a wrapped gaussian distribution for the periodic rotational distribution.

In Example 12, the subject matter of one or any combination of Examples 9-11 optionally includes a controller configured to display a hotspot indicator superimposed on representations of electrodes of the lead on the user interface as the hotspot view.

In Example 13, the subject matter of one or any combination of Examples 9-12 optionally includes a controller configured to display a hotspot indicator that indicates: a longitudinal location of the peak regions on the length of the lead, an angular location of the peak regions about the lead, and width of the peak regions at the length location and the angular location.

In Example 14, the subject matter of one or any combination of Examples 9-13 optionally includes a controller configured to display representations of electrodes of the lead on the user interface, and display the longitudinal distribution and the periodic rotational distribution of the ER signal features on the user interface relative to the representations of electrodes of the lead.

In Example 15, the subject matter of one or any combination of Examples 9-14 optionally includes a stimulus circuit configured to provide electrostimulation to electrodes of a deep brain stimulator (DBS) lead, and the evoked response signals sensed by the sensing circuit include Evoked Resonant Neural Activity (ERNA) signals.

Example 16 includes subject matter (or can be combined with one or any combination of Examples 1-15 to include such subject matter) such as a machine-readable medium including instructions, which when executed by a machine, cause the machine to perform a method comprising delivering, using a controller of the machine, electrostimulation using a stimulus circuit; producing sensed evoked response (ER) signals using the controller and a sensing circuit; identifying one or more ER signal features of the ER signals using a controller; computing a longitudinal distribution of the one or more ER signal features for a longitudinal direction of the lead; computing a rotational distribution of the one or more ER signal features for an angular direction of the lead, wherein the rotational distribution is a periodic rotational distribution; determining a peak region of the computed longitudinal distribution and a peak region of the computed rotational distribution of the sensed ER signals; and presenting the peak regions as a hotspot view of the sensed ER signals on a user interface.

In Example 17, the subject matter of Example 16 optionally includes the machine readable medium including instructions that cause the machine to: detect, using the controller, when the peak region of the computed periodic rotational distribution is within a specified range of a circular boundary of the periodic rotational distribution; compute an angular rotation of the rotational distribution to move the peak region away from the circular boundary; and determine the hotspot view using the computed angular rotation in response to the detecting.

In Example 18, the subject matter of one or both of Examples 16 and 17 optionally includes the machine readable medium including instructions that cause the machine to: compute, using the controller, the periodic rotational distribution as a wrapped gaussian distribution of the ER signal features.

In Example 19, the subject matter of Example 18 optionally includes the machine readable medium including instructions that cause the machine to: compute, using the controller, the longitudinal distribution as a non-periodic gaussian distribution of the ER signal features; display representations of electrodes of the lead using the user interface; and display the wrapped gaussian distribution of the ER signal features and the non-periodic gaussian distribution of the ER signal features on the user interface with the representations of electrodes of the lead.

In Example 20, the subject matter of one or any combination of Examples 16-19 optionally includes the machine readable medium including instructions that cause the machine to to display, using the user interface, representations of electrodes of the lead and a hotspot indicator superimposed on the electrodes.

These non-limiting Examples can be combined in any permutation or combination. This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of an electrical stimulation system, which may be used to deliver deep brain stimulation (DBS).

FIG. 2 illustrates an example of an implantable pulse generator (IPG) in a DBS system.

FIGS. 3A-3B illustrate examples of leads that may be coupled to the IPG to deliver electrostimulation such as DBS.

FIG. 4 illustrates an example of a computing device for programming or controlling the operation of an electrical stimulation system.

FIG. 5 illustrates an example of an electrical therapy-delivery system.

FIG. 6 illustrates an example of a monitoring system and/or the electrical therapy-delivery system of FIG. 5, implemented using an implantable medical device.

FIG. 7 illustrates an example of a system for

FIGS. 8A and 8B illustrate an example of a display of extracted signal features of sensed evoked response (ER) signals

FIG. 9 illustrates an example of distributions computed for extracted ER signal features.

FIG. 10 illustrates an example of a hotspot view for extracted ER signal features.

FIG. 11 illustrates another example of a hotspot view for extracted ER signal features.

FIGS. 12 and 13 illustrate examples of a normal Gaussian distribution and a wrapped Gaussian distribution for ER signal data.

FIG. 14 is a flow diagram of an example of a method of operating a neurostimulation system.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Evoked responses (ERs) may be used to guide the implantation procedure or programming of parameter settings. The ERs may be caused by stimulation (e.g., electrostimulation pulses) that provides evoked potentials, stimulation that provides therapy, and stimulation that can both deliver therapy and provide ERs. Stimulation may be located (1) where placing evoking pulses gets a desired response such as to maximize ERNA, (2) where listening for responses gets a desired response (e.g., maximize ERNA, (3) where placing lead is desired (e.g., best for therapy), and (4) where placing stimulation on the lead is desired (e.g., maximize therapy and/or minimize/counter side effects). Responses may be modulated by the details of the sensing, including amplifier settings, relationships between stimulating and sensing electrodes, natures of stimulating or sensing electrodes including geometry and surface among other factors, and signal processing occurring during and after measurement, including treatment within analogue or digital hardware, firmware, or software. The target may be an anatomical target such as a tissue volume (e.g., STN), a collection of fibers, a sub-region (motor STN; dorsolateral STN), a volume of interest described within or related to a particular patient's brain such as from atlas or aggregate prior information, or a “point” that may be described by optimizing a stimulation location (e.g., one of (1)-(4) above).

Various embodiments may sense ERs produced through electrostimulation and compute a distribution for the ERs across the electrode space. The distributions can be used to map the more effective locations of the stimulation for feedback to the user. The mapping includes determining an evoked response fit onto the electrodes from the distribution. Fitting the distribution onto the electrodes can become challenging for directional leads. Using a periodic or wrapped distribution can improve fitting the ERs to the electrode space of a directional lead. A periodic or wrapped distribution is a continuous distribution with data points that can lie, or are wrapped, on a sphere (e.g., a unit sphere). Using a periodic distribution produces a response fit that is continuous around the lead.

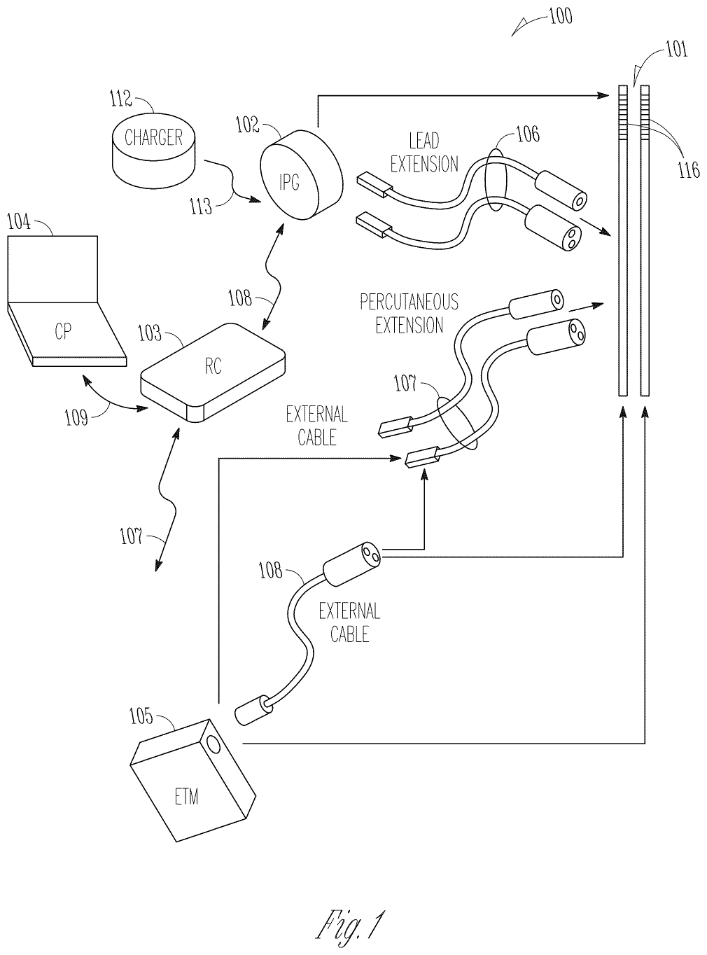

FIG. 1 illustrates an example of an electrical stimulation system 100, which may be used to deliver DBS. The electrical stimulation system 100 may generally include one or more (illustrated as two) implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

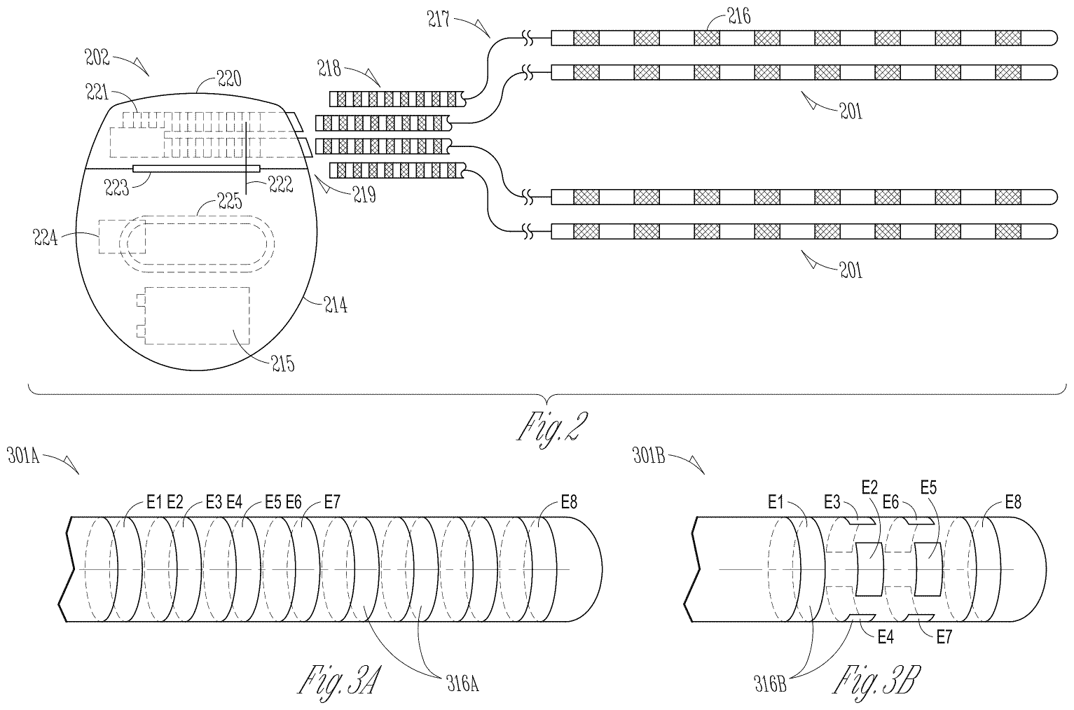

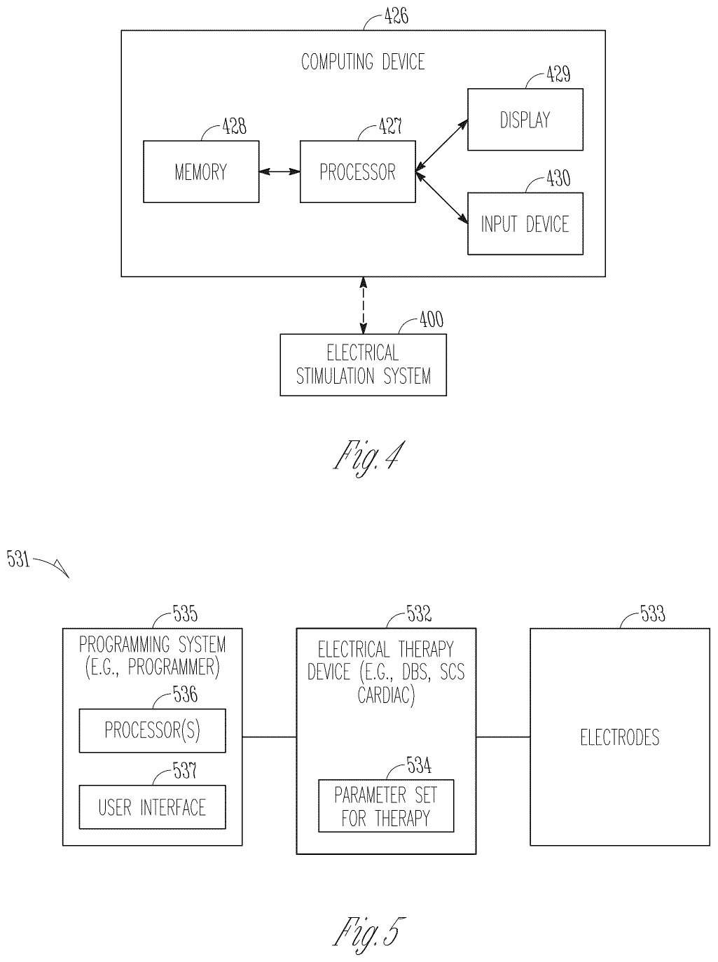

FIG. 2 illustrates an example of an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate examples of leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Additional segmented electrodes can be included between ring electrodes E1 and E8. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. By using a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead 219.

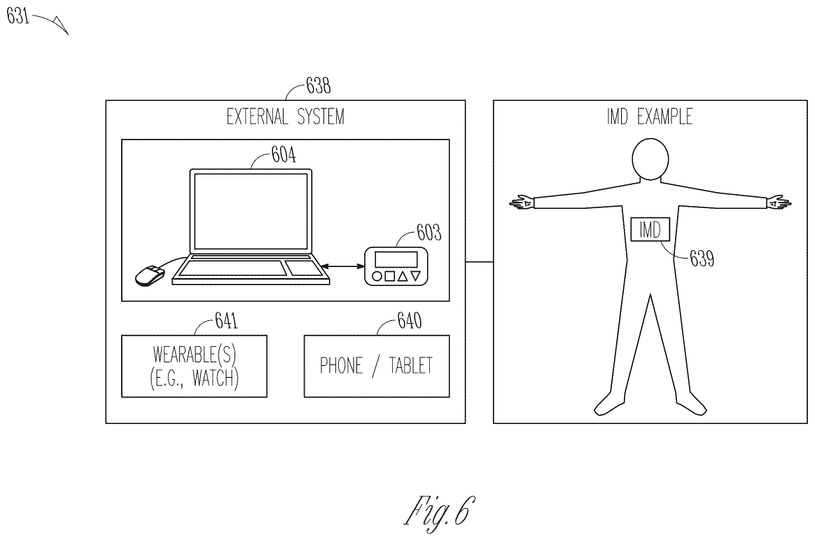

FIG. 4 illustrates an example of a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1.

The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), programmable gate array (PGA), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media, namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

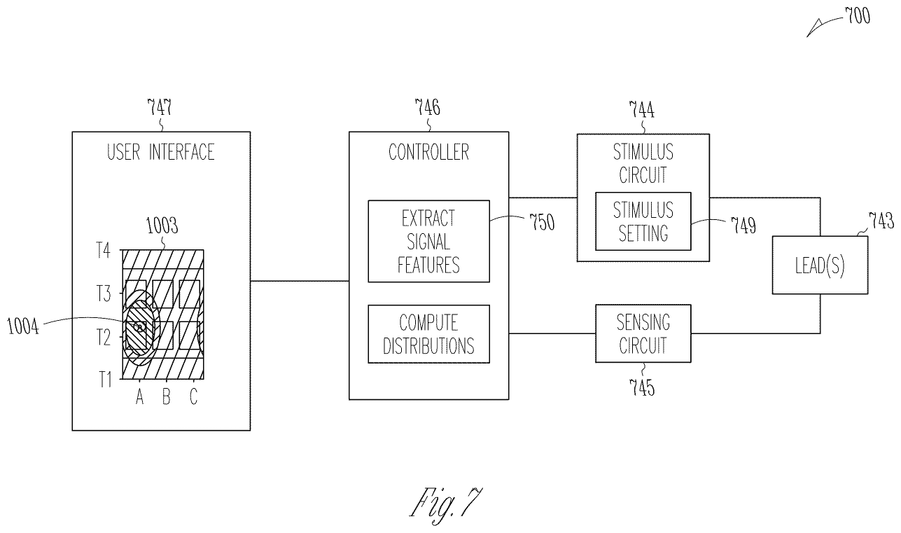

FIG. 5 illustrates an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 6 illustrates an example of the electrical therapy-delivery system of FIG. 5 implemented using an IMD 639. The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

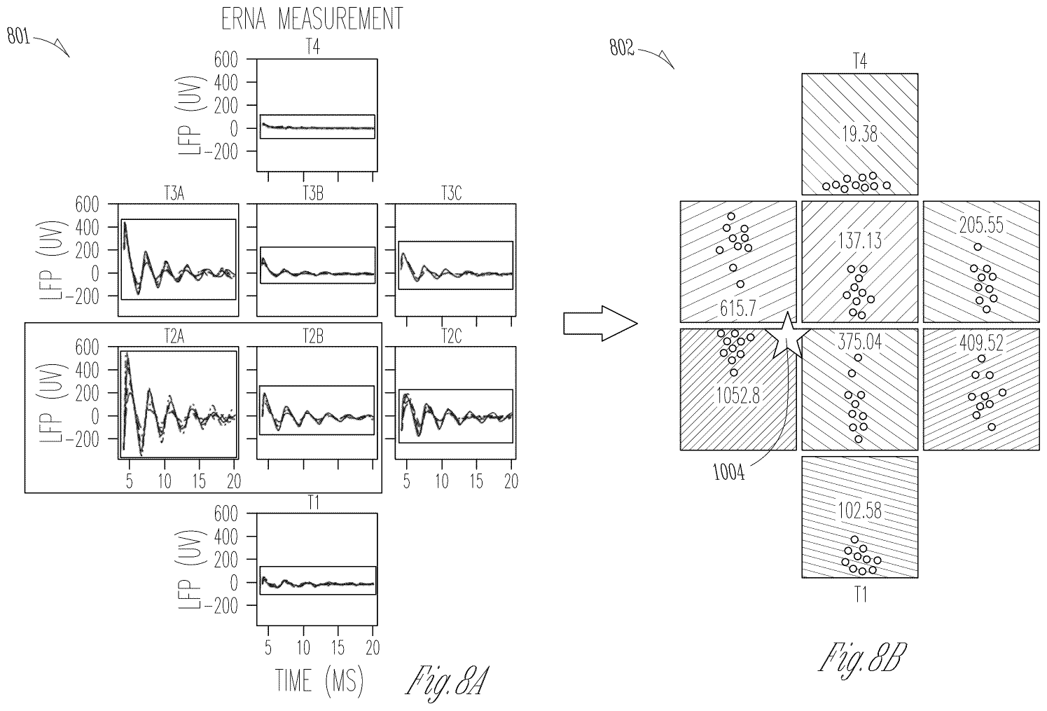

FIG. 7 illustrates an example of a system 700 for mapping ER signal features to an electrode space of a directional lead. The mapping can be referred to as a response fit. The illustrated system 700 includes at least one lead 743 with electrodes, a stimulus circuit 744 operably connected to the lead(s) 743, a sensing circuit 745 operably connected to the lead(s), a controller 746 and a user interface 747. The controller 746 can include a digital signal processor, application specific integrated circuit (ASIC), programmable gate array (PGA), microprocessor, or other type of processor.

The controller 746 is connected to the stimulus circuit 744 and provides stimulation control. The stimulus circuit 744 may be configured by the controller 746 to deliver electrostimulation according to a stimulus setting 749. The stimulus setting 749 may include parameters such as, but not limited to, pulse amplitude pulse width pulse frequency and fractionalization. For example, the controller 746 may program the stimulus settings 749 into the stimulus circuit 744 and may control timing for delivering pulse waves. The controller 746 is also connected to the sensing circuit 745. The sensing circuit 745 senses ER signals produced by the electrostimulation. The ER signals can be sensed at multiple sensing locations based on the configuration of the electrodes of the lead(s) 743. The controller 746 initiates delivery of electrostimulation that will cause ERs. The ER signals are sensed and the controller extracts ER signal features 750 (e.g., ER signal features that were selected by the user) from the sensed signals. The extracted ER signal features may be presented to a user using the user interface 747.

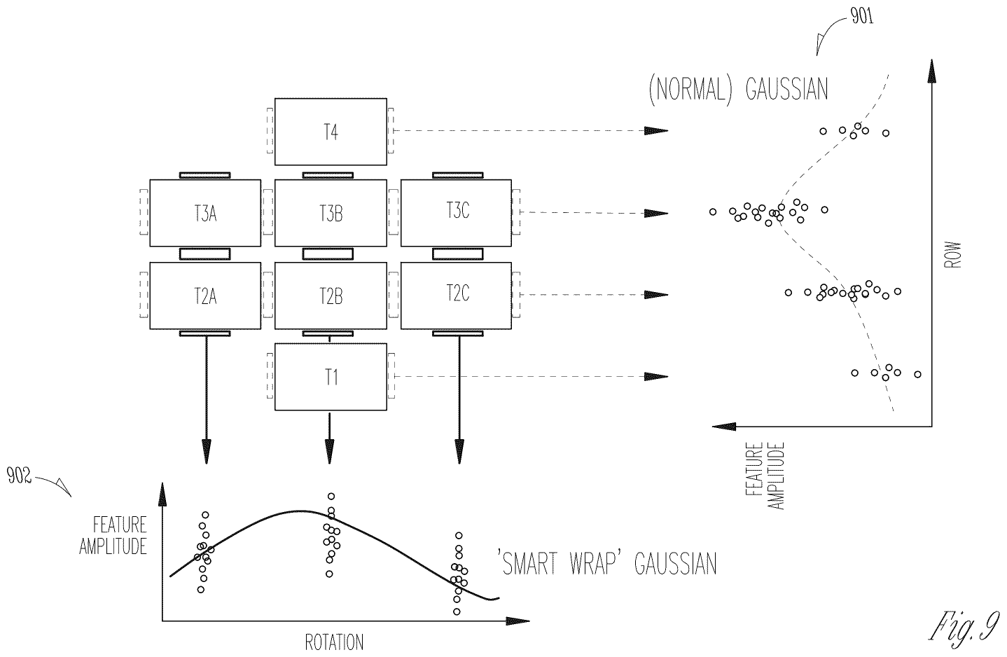

FIGS. 8A and 8B illustrate an example of a display of extracted signal features of sensed ERNA signals. The display can be presented using the user interface 747. Each square in the extracted feature views represents an electrode (or electrode segment for the segmented electrodes for the directional lead). The example illustrates two ring electrodes (labeled T1 and T4) of the lead and two rows of segmented electrodes (labeled T2a, T2b, T2c, and T3a, T3b, T3c) of the directional lead. Other electrode arrangements are possible, such as three rows of segmented electrodes. The raw ERNA measurement data is illustrated in FIG. 8A at 801. The ER measurements may be measured at different locations/electrode vertical locations, and directional locations (a, b, c) at a fixed vertical location (e.g., T3a, T3b, T3c). Further multiple measurements may be taken from each set of one or more electrodes. Multiple measurements from each of the locations.

FIG. 8B shows an extracted signal feature view. One or more signal features may be extracted from the raw ERNA measurement data and presented in an extracted feature view 802 that provide a distribution of extracted feature(s). For example, features of the signals such as amplitude, magnitude, first peak, width, RMS value, and the like may be extracted from the raw ER signals. The controller 746 computes distributions of the extracted signal features from ER signals sensed over the electrode space.

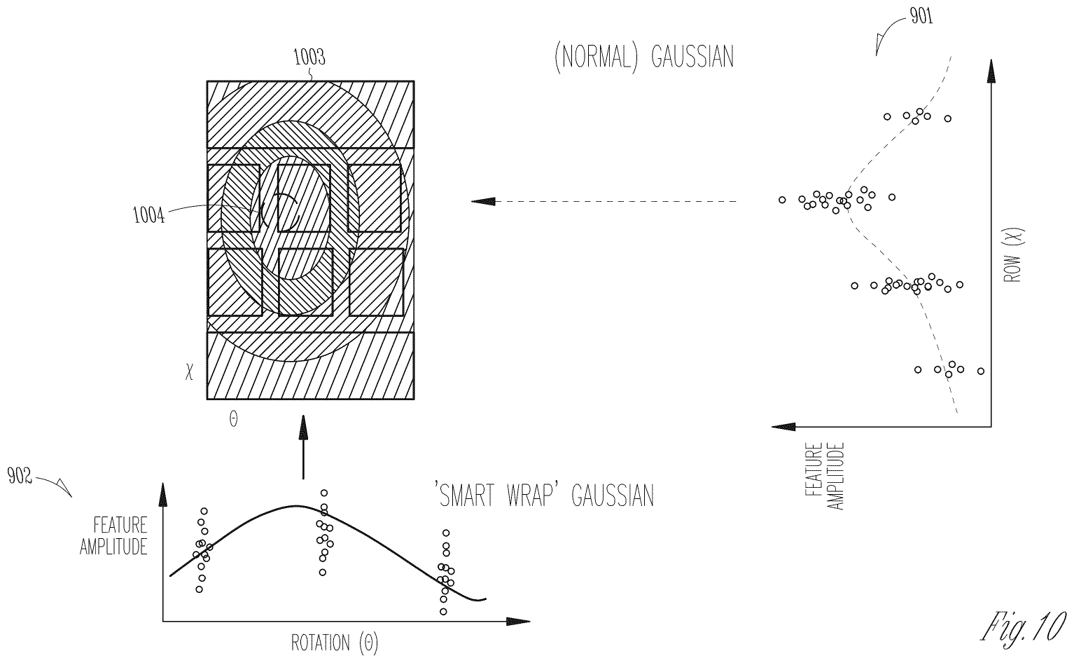

FIG. 9 illustrates an example of distributions computed by the controller 746 for the extracted signal features of FIG. 8B. The extracted amplitude of the ER signals at each electrode are plotted for both the longitudinal locations along the lead and angular locations about the lead. FIG. 9 shows a longitudinal distribution 901 computed by the controller 746 for the longitudinal direction along the length of the lead. In the example of FIG. 9, the computed longitudinal distribution is a normal or Gaussian distribution. The longitudinal distribution fₗ(x) can be calculated as

$${f}_{l} ( x ) = {a}_{l} exp [ \frac{- {( x - {μ}_{l} )}^{2}}{2 {{σ}_{l}}^{2}} ]$$

where aₗ is the height of the peak of the Gaussian distribution, μₗ is the location of the distribution peak along the length of the lead (e.g., an electrode location), and σₗ is the standard deviation or width of the distribution peak.

The controller 746 computes a rotational distribution 902 for an angular direction about the lead. In some examples, the rotational distribution 902 is a non-periodic distribution, (e.g., a non-periodic Gaussian distribution). In other examples, the rotational distribution 902 is a periodic or cyclic distribution that can be referred to as a wrapped fit function. In the example of FIG. 9, the computed rotational distribution is a wrapped Gaussian distribution as the wrapped fit function. Other wrapped fit functions can be used. The rotational distribution fᵣ(x) can be calculated as

$${f}_{r} ( θ ) = {a}_{r} {\sum }_{k = - 10}^{10} exp [ \frac{- {( θ - {μ}_{r} + 2 π k )}^{2}}{2 {{σ}_{r}}^{2}} ]$$

where aᵣ is the height of the peak of the wrapped Gaussian distribution, μᵣ is the angular location of the distribution peak about the lead (e.g., an electrode angle), and σᵣ is the standard deviation or width of the distribution peak.

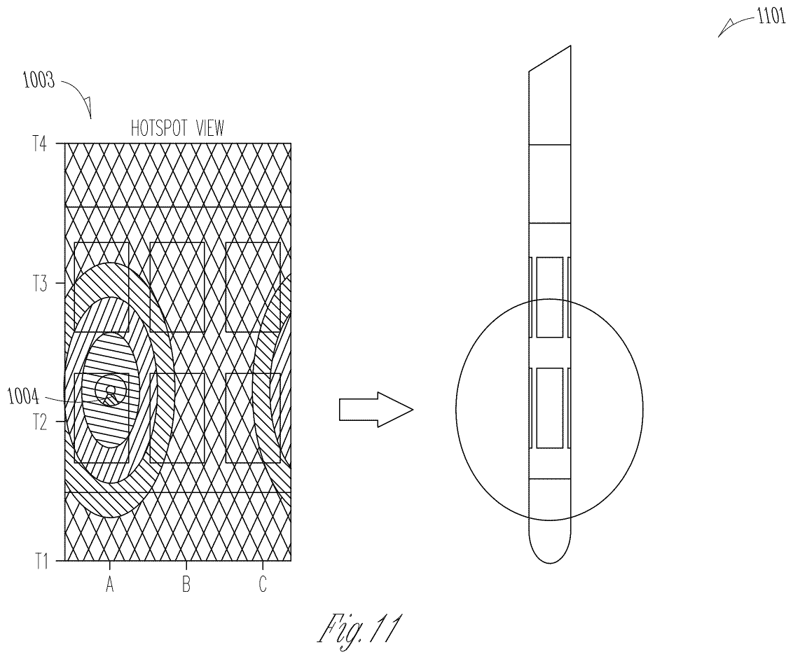

The controller may use the computed distributions of the extracted features 746 to create a hotspot view or hotspot fit of the ERs. FIG. 10 illustrates an example of a hotspot view. The hotspot view is superimposed on an electrode template representing electrodes of the lead, including ring electrodes T1 and T4, and segmented electrodes T2 and T3. The hotpot view may be color coded similar to thermal mapping, where more features in a particular area result in a “hotter” color for the area. The hotpot view 1003 created by the controller 746 can be displayed on the user interface 747. The area with the most features may be displayed as a hotspot indicator 1004. (e.g., a highlight or icon). The hotspot indicator 1004 may correspond to peak regions of the computed distributions. The hotspot indicator 1004 may be positioned to represent the length location and the angular location of the peak regions on the length of the lead. The width of the hotspot indicator 1004 may correspond to the width or standard deviation of the peak regions. In certain examples, a hotspot indicator 1004 is displayed superimposed on the electrodes with the ER signal features as in the example of FIG. 8B. In variations, the hotspot view 1003 may be displayed with the computed distributions with the ER signal features as in the example of FIG. 10. The distributions 901 and 902 may include a best fit line for plotted values for the sensed ERs.

FIG. 11 is an illustration of a hotspot view 1003 with hotspot indicator 1004 that may be presented on the user interface 747. Also illustrated in FIG. 11 is a lead 1101 that shows a representation of the stimulation steering setting corresponding to the results shown in the hotspot view. The user interface 747 may be interactive and may allow the user to accept the stimulation steering state based on the images displayed on the user interface 747.

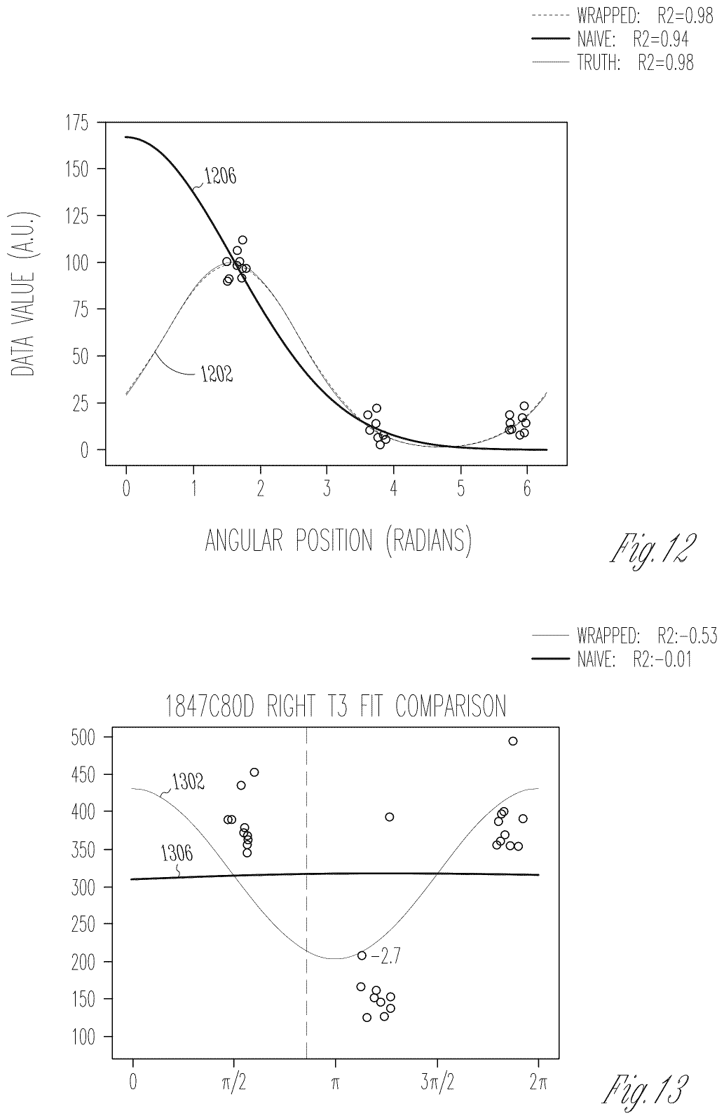

Using a periodic distribution, such as a wrapped Gaussian distribution or other wrapped fit function, for the rotational distribution 902 ensures that the response fit for the hotspot view is continuous around the lead and does not include voids. FIG. 12 illustrates examples of graphs of a non-periodic Gaussian distribution 1206 and a wrapped Gaussian distribution 1202 for the angular direction of a lead. The graphs show that the non-periodic Gaussian distribution 1206 does not account for the periodic nature of the ER data.

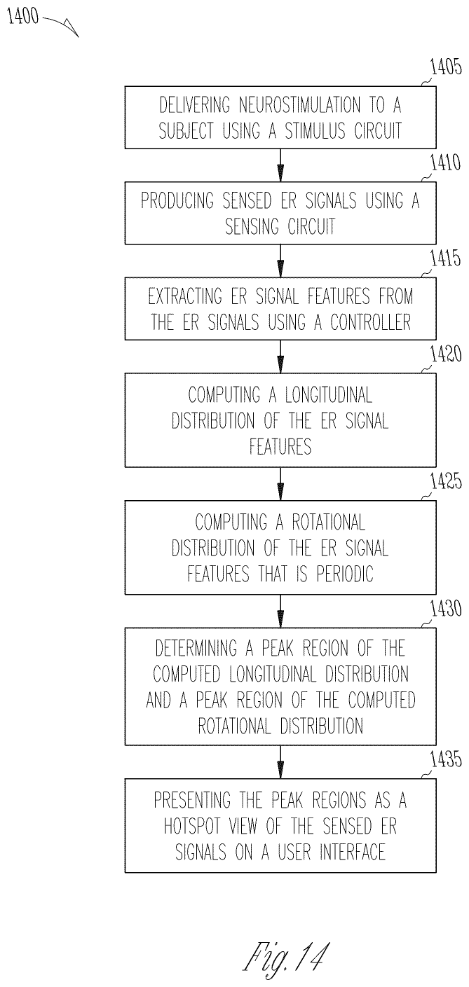

An issue with using a wrapped Gaussian distribution can arise when the peak of the rotational distribution occurs close to a circular boundary such as a zero or 2x boundary. The wrapped Gaussian distribution may have difficulty placing the center of the distribution and finding the best fit line. FIG. 13 illustrates examples of graphs of a non-periodic Gaussian distribution 1306 and a wrapped Gaussian distribution 1302 when the ER signal features have a peak near the 2x boundary.

To address this, the controller 746 can compute an angular rotation of the rotational distribution when the peak of the distribution is within a specified range of a circular boundary of the rotational distribution. This angular rotation moves the peak away from the boundary. A best fit line is then determined for the rotated distribution. The controller 746 rotates the distribution back to the original orientation after the best fit is determined. For instance, if the controller 746 computes a wrapped Gaussian distribution that has a peak near the zero of 2x boundary as in the example of FIG. 13, the controller 746 rotates the ER data of the distribution by x. After determining the best fit, the distribution is rotated back to the original angular orientation to determine the hotspot view.

For completeness, FIG. 14 is a flow diagram of an example of a method 1400 of operating a neurostimulation system (e.g., the system of FIG. 7). At block 1405, electrical neurostimulation is delivered to a patient to produce ERs. The stimulation is provided using a stimulus circuit or other electrostimulator connected to a directional lead. The ERs are useful to provide feedback for programming the steering state of the neurostimulation. The neurostimulation may sweep the electrode space of the lead and use different electrode configurations to deliver the neurostimulation energy.

At block 1410, ER signals corresponding to the produced ERs are sensed by the system using a sensing circuit connected to the directional lead. The ER signals can be sensed using the same electrodes used to deliver the neurostimulation energy or sensed using different electrodes. At block 1415, ER signal features are extracted using a controller operatively coupled to the sensing circuit. The ER signal features can be one ER signal feature extracted from multiple ER signals, multiple ER signal features extracted from one ER signal, or multiple ER signal features extracted from multiple ER signals. The result is a set of extracted signal features for locations along the lead and for directions about the lead. FIG. 8A illustrates an example of ER signals recorded for different locations and directions of the lead. The system can extract one or more ER signal features from the recorded signals.

At block 1420, the system computes a longitudinal distribution of an ER signal feature in the longitudinal direction along the lead. In an example intended to non-limiting, the longitudinal distribution can be a non-periodic Gaussian distribution along the length of the lead of the peak amplitude of the sensed ER signals. At block 1425, the system computes a rotational distribution of an ER signal feature in the angular direction about or around the lead. The rotational distribution is periodic (e.g., periodic about a unit circuit or sphere). The rotational distribution can be a wrapped Gaussian distribution.

At block 1425, the peak regions of the distributions are determined. The peak regions have a longitudinal location on the lead, an angular location on the lead, and a width. At block 1430, the system determines a hotspot view or hotspot fit for the ER signal feature. The computed ER signal distributions can be superimposed onto a two-dimensional representation of the electrode space. Color coding can be used to show the peak regions and a hotspot view on the two-dimensional representation of the electrode space. The hotspot view can be displayed on the user interface of the system. Outlier signal data can be handled by other methods, or the outlier data can be fit to the computed distributions.

The ER signal feature displayed may be a feature of interest selected by a user. In some examples, the user may change the signal feature of interest. The system may recompute the distributions and determine a new hotspot view for the user interface according to the selection by the user.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A computer-implemented method of operating a neurostimulation system when connected to electrodes of a lead, the method comprising:
delivering neurostimulation to a subject using a stimulus circuit;
producing sensed evoked response (ER) signals using a sensing circuit;
extracting ER signal features from the ER signals using a controller;
computing a longitudinal distribution of the ER signal features for a longitudinal direction of the lead using the controller;
computing a rotational distribution of the ER signal features for an angular direction of the lead using the controller, wherein the rotational distribution is a periodic rotational distribution;
determining a peak region of the computed longitudinal distribution and a peak region of the computed periodic rotational distribution of the sensed ER signals; and
presenting the peak regions as a hotspot view of the sensed ER signals on a user interface.

2. The method of claim 1, including:
detecting when the peak region of the computed periodic rotational distribution is within a specified range of a circular boundary of the periodic rotational distribution;
computing an angular rotation of the periodic rotational distribution to move the peak region farther away from the circular boundary; and
determine the hotspot view using the computed angular rotation in response to the detecting.

3. The method of claim 1, wherein the computing the rotational distribution includes computing a wrapped fit function of the ER signal features.

4. The method of claim 3, wherein the computing the wrapped fit function includes computing a wrapped gaussian distribution and wherein the computing the longitudinal distribution includes computing a non-periodic gaussian distribution of the ER signal features.

5. The method of claim 1, wherein presenting the hotspot view includes displaying a hotspot indicator superimposed on representations of electrodes of the lead on the user interface.

6. The method of claim 1, wherein the displaying the hotspot indicator includes displaying:
a longitudinal location of the peak regions on the length of the lead;
an angular location of the peak regions about the lead; and
a width of the peak regions at the length location and the angular location.

7. The method of claim 1, including:
displaying representations of electrodes of the lead on the user interface; and
displaying the longitudinal distribution and the periodic rotational distribution of the one or more extracted signal features on the user interface with the representations of electrodes of the lead.

8. The method of claim 1, wherein the stimulus circuit is configured to provide electrostimulation to electrodes of a deep brain stimulator (DBS) lead, and the evoked response signals produced by the electrostimulation include Evoked Resonant Neural Activity (ERNA) signals.

9. A system, comprising:
a stimulus circuit configured to provide electrostimulation to a neural target of a patient via electrodes on a lead;
a sensing circuit configured to sense, at a plurality of sensing locations, evoked response (ER) signals produced by the electrostimulation;
a user interface; and
a controller operably connected to the stimulus circuit, the sensing circuit and the user interface, and configured to:
initiate delivery of the electrostimulation to the electrodes;
identify ER signal features of the sensed ER signals;
compute a longitudinal distribution of the ER signal features for a longitudinal direction of the lead;
compute a rotational distribution of the ER signal features for an angular direction of the lead, wherein the rotational distribution is a periodic rotational distribution; and
display peak regions of the longitudinal distribution and the periodic rotational distribution as a hotspot view of the sensed ER signals on the user interface.

10. The system of claim 9, wherein the controller is configured to:
determine when the peak region of the computed periodic rotational distribution is within a specified range of a circular boundary of the periodic rotational distribution; and
compute an angular rotation of the periodic rotational distribution to adjust the peak region for the periodic rotational distribution.

11. The system of claim 9, wherein the controller is configured to compute a non-periodic gaussian distribution for the longitudinal distribution and compute a wrapped gaussian distribution for the periodic rotational distribution.

12. The system of claim 9, wherein the controller is configured to display a hotspot indicator superimposed on representations of electrodes of the lead on the user interface as the hotspot view.

13. The system of claim 12, wherein the hotspot indicator indicates:
a longitudinal location of the peak regions on the length of the lead;
an angular location of the peak regions about the lead; and
a width of the peak regions at the length location and the angular location.

14. The system of claim 9, wherein the controller is configured to:
display representations of electrodes of the lead on the user interface; and
display the longitudinal distribution and the periodic rotational distribution of the ER signal features on the user interface relative to the representations of electrodes of the lead.

15. The system of claim 9, wherein the stimulus circuit is configured to provide electrostimulation to electrodes of a deep brain stimulator (DBS) lead, and the evoked response signals sensed by the sensing circuit include Evoked Resonant Neural Activity (ERNA) signals.

16. A non-transitory machine-readable medium including instructions, which when executed by a machine, cause the machine to perform a method comprising:
delivering, using a controller, electrostimulation using a stimulus circuit;
producing sensed evoked response (ER) signals using the controller and a sensing circuit;
identifying one or more ER signal features of the ER signals using a controller;
computing a longitudinal distribution of the one or more ER signal features for a longitudinal direction of the lead;
computing a rotational distribution of the one or more ER signal features for an angular direction of the lead, wherein the rotational distribution is a periodic rotational distribution;
determining a peak region of the computed longitudinal distribution and a peak region of the computed rotational distribution of the sensed ER signals; and
presenting the peak regions as a hotspot view of the sensed ER signals on a user interface.

17. The non-transitory machine-readable medium of claim 16, further including instructions that cause the machine to:
detect, using the controller, when the peak region of the computed periodic rotational distribution is within a specified range of a circular boundary of the periodic rotational distribution;
compute an angular rotation of the rotational distribution to move the peak region away from the circular boundary; and
determine the hotspot view using the computed angular rotation in response to the detecting.

18. The non-transitory machine-readable medium of claim 16, further including instructions that cause the machine to compute, using the controller, the periodic rotational distribution as a wrapped gaussian distribution of the ER signal features.

19. The non-transitory machine-readable medium of claim 18, further including instructions that cause the machine to:
compute, using the controller, the longitudinal distribution as a non-periodic gaussian distribution of the ER signal features;
display representations of electrodes of the lead using the user interface; and
display the wrapped gaussian distribution of the ER signal features and the non-periodic gaussian distribution of the ER signal features on the user interface with the representations of electrodes of the lead.

20. The non-transitory machine-readable medium of claim 16, further including instructions that cause the machine to display, using the user interface, representations of electrodes of the lead and a hotspot indicator superimposed on the electrodes.
